(19) United States
(12) Patent Application Publication
(10) Pub. No.: US 2018/0348104 A1
(43) Pub. Date: Dec. 6, 2018

(54) NON-HAZARDOUS OPTICAL CLEARING OF BIOLOGICAL SAMPLES

(71) Applicant: Universitaet Duisburg-Essen, Essen (DE)

(72) Inventor: Anika KLINGBERG, Essen (DE)

(73) Assignee: Universitaet Duisburg-Essen, Essen (DE)

(21) Appl. No.: 15/779,062

(22) PCT Filed: Nov. 30, 2016
(86) PCT No.: PCT/EP2016/079286

§ 371 Date: May 24, 2018

(30) Foreign Application Priority Data
Dec. 1, 2015 (EP) 15197257.7

(51) Int. Cl. G01N 1/34; G01N 33/483

(52) U.S. Cl. CPC G01N 1/34; G01N 33/4833

(57) ABSTRACT

The invention provides a method for optical clearing of biological samples utilizing a composition comprising a cinnamate ester. The invention further provides a kit suitable for performing said method and the use of said composition for optical clearing of biological samples.

### BACKGROUND OF THE INVENTION

The inherent three-dimensional structure of biological specimens like tissues and organs gives rise to the need for volumetric (three-dimensional) optical imaging to acquire complete structural information. Optical imaging of thick tissues or whole organs is however limited by lateral light scattering that arises from the heterogeneity of refractive indices of the various sample components.

Major advances in the field have been accomplished by the development of clearing techniques that focus on equilibrating the refractive index throughout a sample (refractive index matching) to reduce inhomogeneities in light scatter. These methods can generally be subdivided into two basic approaches: solvent-based clearing and aqueous-based clearing. A comprehensive overview of available clearing techniques is provided in Table 1 of D. S. Richardson & J. W. Lichtman, Cell 162(2):246-257 (2015).

Solvent-based clearing techniques require an initial dehydration that is most commonly accomplished either by the use of alcohol (ethanol, methanol) or tetrahydrofuran (THF) (K. Becker et al., PLoS ONE 7(3):e33916 (2012); A. Ertürk et al., Nat. Protoc. 7(11):1983-1995 (2012)). Following dehydration, clearing of the samples by refractive index matching is achieved through the use of appropriate solvents. Methylsaliciate, benzyl alcohol, benzyl benzoate, dichlormethane and dibenzylether have all been used as final clearing solutions (K. Becker et al., PLoS ONE 7(3):e33916 (2012); H. U. Dodt et al., Nat. Methods 4(4):331-336 (2007); A. Ertürk et al., Nat. Protoc. 7(11):1983-1995 (2012); N. Renier et al., Cell 159(4):896-910 (2014), W. Spalteholz (1914): Über das Durchsichtigmachen von menschlichen und tierischen Praparaten und seine theoretischen Bedingungen, nebst Anhang: Über Knochenfarbung, S. Hirzel; H. Steinke and W. Wolff, Ann. Anat. 183(1):91-95 (2001)). From here it is apparent that available solvent-based clearing techniques involve handling of hazardous reagents, such as e.g. THF, methylsaliciate, benzyl alcohol, benzyl benzoate, dichlormethane or dibenzylether. A further limitation of solvent-based clearing techniques is the often observed quenching of fluorescent protein emission, in particular when using alcohols as dehydration solutions (H. U. Dodt et al., Nat. Methods 4(4):331-336 (2007), K. Becker et al., PLoS ONE 7(3):e33916 (2012)). Such shortcomings are not encountered with most aqueous-based clearing techniques like the recently developed See DB (M. T. Ke et al., Nat. Neurosci. 16(8):1154-1161 (2013), Scale (H. Hama, Nat. Neurosci. 14(11):1481-1490 (2011) and W02011/111876) and FocusClear (A. S. Chiang et al., Proc. Natl. Acad. Sci. 99(1):37-42 (2002) and US Patent No. 6,472,216) or 3DISCO (A. Ertürk et al., Nat. Protoc. 7(11):1983-1995 (2012)). The application of these techniques is, however, restricted to relative thin tissue samples (composed of a few cell layers) and/or suffers from long incubation steps making their use time-consuming.

### SHORT DESCRIPTION OF THE INVENTION

It has now been found that a solution containing a cinnamic ester, such as ethyl cinnamate, is suitable as clearing solution in a method of optical clearing of biological samples. Such a solution is safe (i.e. nonhazardous), can preserve fluorescence of the biological samples and leads to effective clearing within only a few days. The invention thus provides

(1) a method of optical clearing of a biological sample comprising the steps of

(i) dehydrating the biological sample and

(ii) clearing the dehydrated biological sample by incubation in a clearing composition comprising a cinnamic ester;

(2) a preferred embodiment of the method of (1) above, wherein the dehydrating step (i) comprises the successive application of dehydration compositions having increasing concentrations of ethanol;

(3) a kit for optical clearing of a biological sample as defined in (1) or (2) above, the kit comprising (i) a series of dehydration compositions of aqueous ethanol having increasing concentrations of ethanol and (ii) a clearing composition according to (1) above; and

(4) the use of a clearing composition as defined in (1) above for optical clearing of biological samples.

### SHORT DESCRIPTION OF THE FIGURES

FIG. 1: Optical Clearing of soft and hard tissues via Ethanol-Ethyl-Cinnamate.

FIG. 2: Whole organ quantitative biology (qWOB) on NTN kidneys. LSFM of specifically stained endothelial structures allows 3-D reconstruction of whole kidneys.

### DETAILED DESCRIPTION OF THE INVENTION

The invention provides a method as described in (1) and (2) above suitable for optical clearing of biological samples, such as tissues and organs (e.g. brain, lung, heart, kidney, liver, spleen or bone) including thicker tissue samples (composed of multiple cell layers).

The dehydrating step (i) of the method may comprise the successive application of dehydration compositions containing aqueous ethanol with ethanol in increasing concentrations. The ethanol concentrations used depend on the type of biological sample. In an embodiment of the invention, ethanol concentrations from about 30 to about 100% by volume (vol.-%) are used, preferably ethanol concentrations of about 30%, about 50%, about 70% and about 100% are successively used. In another embodiment, only ethanol concentrations from about 50 to about 100%, e.g. about 50%, about 70% and about 100% are used.

The treatment steps with a certain ethanol concentration may be repeated, i.e. the identical ethanol concentration may be used in two successive applications. The duration of the dehydrating step and in particular the time per single application step depends on the biological sample. The application times per single application step are between about 4 and about 12 hours.

The dehydration compositions may further comprise one or more surfactants, including nonionic surfactants, e.g. polysorbate surfactants such as polysorbate 20 (Tween® 20), polysorbate 80 (Tween® 80), Polyethylene glycol tert-octylphenyl ether (TritonX-100), glycerol or 1-Thioglycerol. In a preferred embodiment of the invention, the dehydration compositions comprise the surfactant(s) in a concentration from about 0.1 to about 5 vol.-%, preferably about 0.5 to about 3%, most preferably about 2%. Suitable cinnamic esters for the clearing solution utilized in step (ii) include compounds of Formula (I)

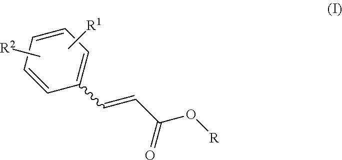

wherein

R is a C₁₋₆ alkyl group which may be substituted with 1 to 3 groups R¹, and

R¹ and R² are independently selected from hydrogen, halogen, C₁₋₃ alkyl and C₁₋₃ alkoxy.

In a preferred embodiment R is a C₁₋₃ alkyl group and R¹ and R² are hydrogen or methyl.

The cinnamic ester may have E or Z (trans or cis) configuration, mixtures of both configurations are also applicable. Preferred however are cinnamic esters in the more abundant trans configuration. A particularly preferred cinnamic ester is ethyl trans-cinnamate.

It is preferred that the clearing composition comprises from about 10 to about 100 vol.-% , preferably about 50 to about 100% cinnamic ester, the remainder being an organic solvent, preferably an optically feasible, inert organic solvent with a refractive index of about 1.5. Particularly preferred is that the clearing composition consists of ethyl trans-cinnamate.

It is furthermore preferred that the biological sample harbors fluorescence, said fluorescence being derived from a fluorescent protein or staining of the biological sample with a fluorophore-coupled binding ligand prior to the optical clearing. Moreover, in the method of the invention the fluorescence is preserved after clearing. The fluorescence of a biological sample can stem from a fluorescent protein like GFP, YFP and other known derivatives thereof. Fluorescence of a biological sample can also result from staining of the biological sample with a fluorophore-coupled antibody. Examples for fluorophore/antibody pairs include all antibodies coupled to AF-dyes such as CD31-AF647, CD45-AF647, SiglecF-AF594, CD11c-AF647, but also fusions to eGFP, eYFP or tdTomato.

The method may further comprise optical imaging of the cleared biological sample, including its fluorescence. Optical imaging techniques employed in the method include lightsheet microscopy, confocal microscopy and two-photon microscopy.

The invention further provides a kit as described in (3) above suitable for optical clearing of biological samples according to the method of the invention. The kit comprises a series of dehydration compositions of aqueous ethanol having increasing concentrations of ethanol and a clearing composition as defined hereinbefore. Such a “series of dehydration compositions” refers to at least two dehydration compositions with different concentrations of ethanol. The kit may however comprise more than two dehydration compositions with different concentrations of ethanol, e.g. the series may consist of three, four, five or more dehydration compositions. The dehydration compositions as well as the clearing composition included in the kit of the invention are preferable those described above in the context of the method of aspects (1) and (2) of the invention. The invention will be further described in the following Examples.

### EXAMPLES

Material and Methods

1. Antibody staining and sample fixation: 8 to 12 weeks old female mice (CD11c-eYFP, CX3CR-eGFP, Catchup^{ivm}) were injected i.v. with 10 μg per mouse CD31-AF647 (Biolegend, Cat# 102516) in PBS (total volume 150 μl) and killed by CO₂ 10 min after injection.

Immediately after killing mice were transcardially perfused with 15 ml of cold PBS/5 mM EDTA and perfusion fixed with 15 ml of cold 4% PFA, pH=7.4. After perfusion organs were removed and post fixed in cold 4% PFA pH=7.4 for 2-4 h (according to the different organs→bones 4 h, all other organs 2 h) at 4-8° C.

2. Sample dehydration and Clearing: According to the organs different incubation times and Ethanol (EtOH) concentrations (in vol.-%; all with adjusted pH=9; surfactant in vol.-%) for sample dehydration are used:

| Organ | 30% EtOH | 50% EtOH | 70% EtOH | 2x 100% EtOH |
|---|---|---|---|---|
| Brain | +2% Tween20 | +2% | +2% Tween20 | +2% Tween20 |
| | 12 h | Tween20 | 12 h | 12 h |
| | | 12 h | | |
| Lung | — | 4 h | 4 h | 4 h |
| Heart | — | 4 h | 4 h | 4 h |
| Kidney | — | 4 h | 4 h | 4 h |
| Liver | — | 4 h | 4 h | 4 h |
| Spleen | — | 4 h | 4 h | 4 h |
| Bones | — | 12 h | 12 h | 12 h |

Incubation of samples is performed at 4-8° C. in gently shaking 5 ml tubes. For optimal tissue dehydration the incubation with 100% EtOH has to be done twice to remove all left over water molecules.

For brain dehydration high concentrations of Tween20 (2%) and longer incubation times of 12 h are essential to prevent/avoid tissue shrinking and to guarantee deep tissue penetration of EtOH via tissue permeabilization. As the mineralized compact bone decelerates the dehydration process longer incubation times of 12 h per concentration are required.

After dehydration the samples are transferred to ethyl trans-cinnamate (Sigma Aldrich, Cat# 112372) or dibenzylether (Sigma Aldrich, Cat# 33630) and incubated under gently shaking at room temperature (as the freezing/melting point/range of ethyl trans-cinnamate (ECi) is 6-8° C.) until they become transparent. Dependent on the different organs the incubation times vary from 2-6 h.

3. Light-Sheet Microscopy: To image whole organs a LaVisionBioTec UltraMicroscope with an Olympus MVX10 zoom microscope body, an Andor Neo sCMOS camera and an optical magnification range of 1.26 x-12.6 x was used. For YFP and GFP excitation an 488 nm Optically Pumped Semiconductor Laser (OPSL) and for Alexa647 excitation a 647 nm Diode Laser was used. Emitted wavelengths were detected with specific detection filters: FITC 525/50 for eGFP, TagYFP 545/30 for eYFP and Cy5 680/30 for Alexa647. As the Excitation Optics/sheet illumination of the LaVisionBioTec Ultramicroscope provide a sheet thickness of 5-40 μm the z-step size was set to 5 μm for all measurements. The optical zoom factor varied from 1.6 x-10 x.

4. Confocal/2-Photon microscopy: For high magnification imaging of the samples a Leica TCS SP8 fully automated epifluorescence confocal microscope with AOTF and AOBS scanoptics, gated HyD detection, 2-Photon (MP) and compact OPO on a DM6000 CFS basic frame was used.

5. Data analysis: For data analysis ImageJ (Image Processing and Analysis in Java, http://imagej.nih.gov/ij/) and IMARIS 8.1.2, Bitplane were used. 3-D rendering of Light-Sheet data as well as 2-Photon data was performed via IMARIS software.

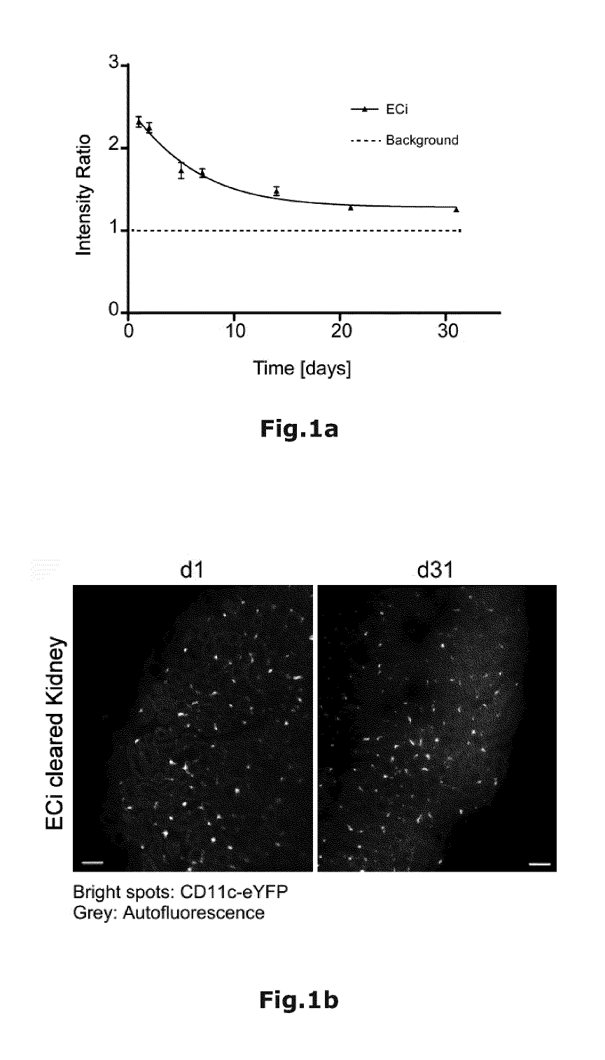

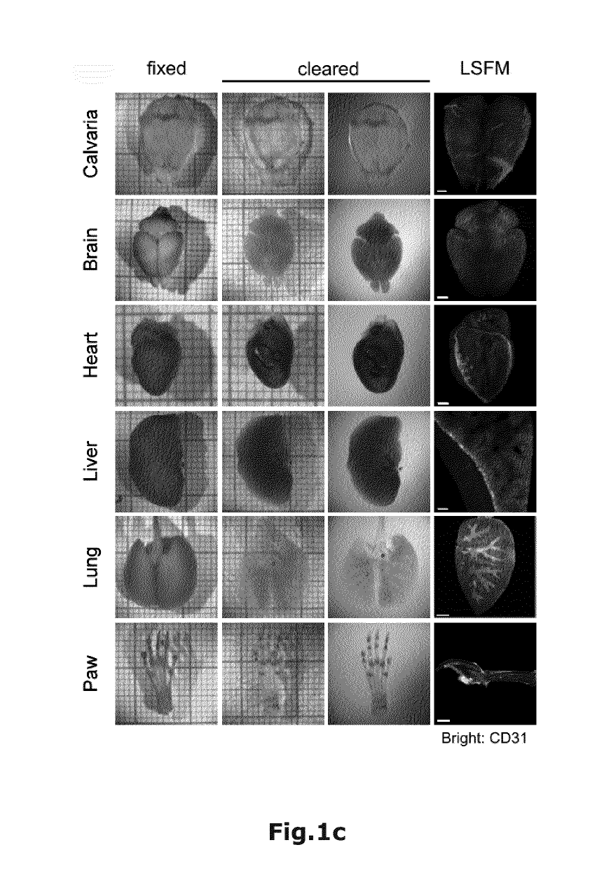

FIG. 1: Optical Clearing of soft and hard tissues via Ethanol-Ethyl-Cinnamate.

(a) In combination with pH-adjusted sample dehydration by Ethanol, Ethyl-cinnamate (ECi) preserves fluorescence of proteins for at least one month, shown via long term measurements of endogenously expressed YFP in murine kidneys. (b) Exemplary lightsheet fluorescence microscopy (LSFM) data of long time measurements showing EYFP positive cells (bright spots) in kidneys of CD11c-EYFP mice (tissue-autofluorescence, grey) at 1 day (d1) and 31 days (d31) post Ethanol-ECi clearing (Scalebars=50 μm). (c) Optical clearing via ECi is not exclusively working for soft tissues as brain, heart, liver and lung but is also successfully clearing hard tissues such as calvarial bone or long bones. In addition to fluorescence protein (FP) preservation fluorescence labeling by antibodies resists the clearing procedure as shown via endothelial specific staining (CD31, bright). (Scalebars=1000 μm, liver and paw Scalebars=100 μm).

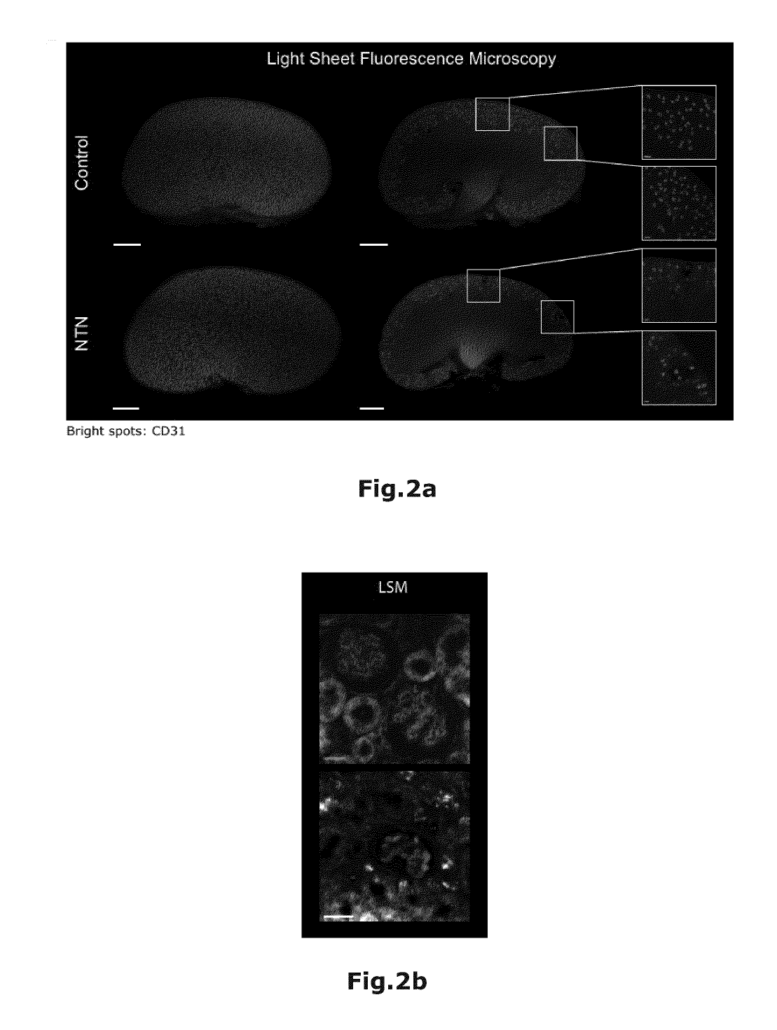

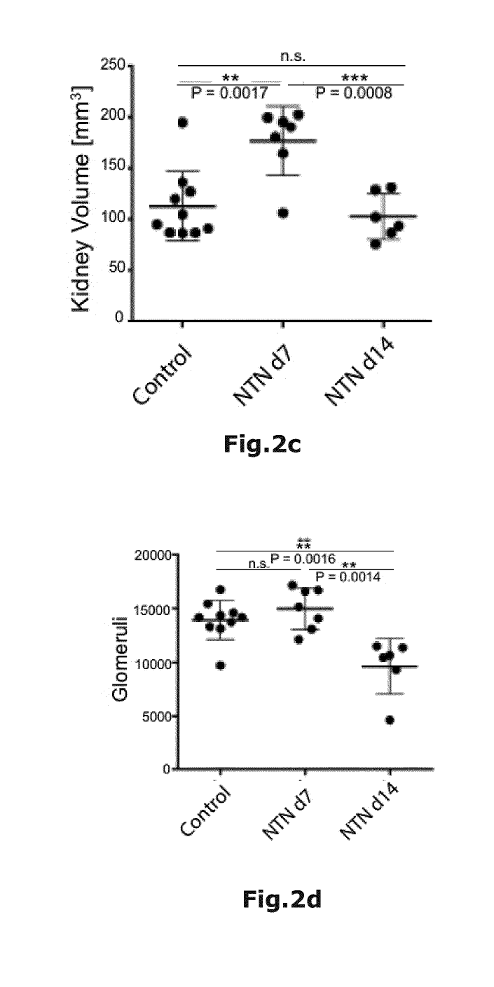

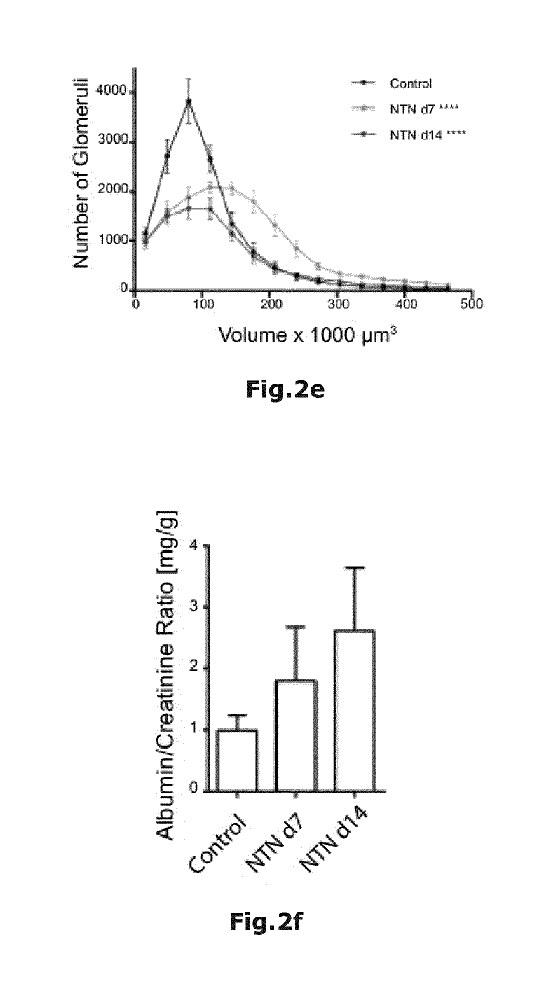

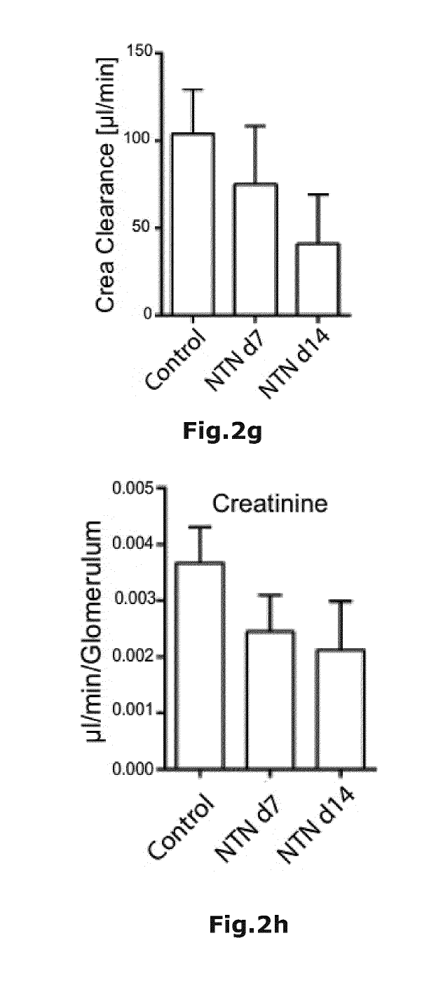

FIG. 2: Whole organ quantitative biology (qWOB) on NTN kidneys.

LSFM of specifically stained endothelial structures (CD31, bright spots) allows 3-D reconstruction of whole kidneys. (a) 3-D reconstructions of NTN kidneys with crescentic glomerulonephritis (cGN) at 14 days post NTN induction (d14) show lower glomerular density while 2-D optical sections reveal CD31 negative areas of corrupted glomeruli and the surrounding vasculature (white boxes, Scalebars=50 μm) compared to healthy controls. (b) Enhanced view of kidney structure via combined confocal and 2-photon laser scanning microscopy (LSM). Compared to control NTN mice show decreased glomerular size, endothelial damage in their capillaries and increased tissue fibrosis around damaged glomeruli (Scalebars=20 μm). (c) Based on whole organ images the total kidney volume was calculated and shows the typical swelling of NTN kidneys at 7 days post NTN induction (d7) and declining kidney size down to control levels at d14 (**P<0.05, ***P<0.001, two-tailed, unpaired t-test). (d) The total numbers of glomeruli per kidney were quantified with a fully automated image processing algorithm showing a highly significant loss of glomeruli at d14 of NTN compared to d7 and controls (**P<0.05, two-tailed, unpaired t-test). (e) Distribution of glomerular volumes of NTN mice at d7 and d14 relative to the distribution in healthy controls (****P<0.0001, chi-square test). For the generation of (d) and (e) a total number of 302,023 glomeruli from 23 kidneys were individually counted and sized. (f) Daily urine analysis shows loss of glomerular filtration functionality, indicated by increasing albumin/creatinine ratio and (g) decreasing Creatinine clearance (non-significant, two-tailed, unpaired t-test). (h) Based on qWOB quantification of glomeruli the Creatinine clearance efficiency per glomerulus was calculated, showing the decreasing clearance efficiency at d7 and d14 compared to untreated mice, (non-significant, two-tailed, unpaired t-test). For (f)-(h) data of n=2 control animals and n=3 NTN treated animals each d7 and d14 were analyzed.

1.-15. (canceled)

16. A method of optical clearing of a biological sample comprising the steps of
(i) dehydrating the biological sample and
(ii) clearing the dehydrated biological sample by incubation in a clearing composition comprising a cinnamic ester.

17. The method of claim 16, wherein the dehydrating step (i) comprises the successive application of dehydration compositions of aqueous ethanol having increasing concentrations of ethanol.

18. The method of claim 17, wherein the ethanol concentration of the dehydration compositions ranges from 30% to 100 vol-%.

19. The method of claim 17, wherein the dehydration compositions further comprise a surfactant.

20. The method of claim 19, wherein the dehydration compositions comprise the surfactant at a concentration from 0.5 to 5 vol.-%.

21. The method of claim 19, wherein the surfactant is a nonionic surfactant.

22. The method of claim 16, wherein the clearing composition comprises a cinnamic ester of formula (I) 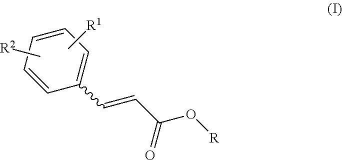
wherein
R is a C₁₋₆ alkyl group which may be substituted with 1 to 3 groups R¹, and
R¹ and R² are independently selected from the group consisting of hydrogen, halogen, C₁₋₃ alkyl and C₁₋₃ alkoxy.

23. The method of claim 22, wherein in the cinnamic ester of formula (1) R is a C₁₋₃ alkyl group and R¹ and R² are independently selected from the group consisting of hydrogen and methyl.

24. The method of claim 22, wherein the cinnamic ester is ethyl trans-cinnamate.

25. The method of claim 16, wherein the clearing composition comprises from 10 to 100 vol.-% of cinnamic ester, the remainder being an optically feasible, inert organic solvent with a refractive index of about 1.5.

26. The method of claim 16, wherein the biological sample harbors fluorescence, said fluorescence being derived from a fluorescent protein or staining of the biological sample with a fluorophore-coupled binding ligand prior to the optical clearing.

27. The method of claim 16 further comprising optical imaging of the cleared biological sample.

28. A kit for optical clearing of a biological sample according to the method of claim 16, the kit comprising
(i) a series of dehydration compositions of aqueous ethanol having increasing concentrations of ethanol, and
(ii) a clearing composition comprising a cinnamic ester.

29. The kit of claim 28, wherein the ethanol concentration of the dehydration compositions ranges from 30% to 100 vol-%.

30. The kit of claim 29, wherein the dehydration compositions further comprise a surfactant.

31. The kit of claim 29, wherein the dehydration compositions comprise the surfactant at a concentration from 0.5 to 5 vol.-%.

32. The kit of claim 28, wherein the clearing composition comprises a cinnamic ester of formula (I)
wherein 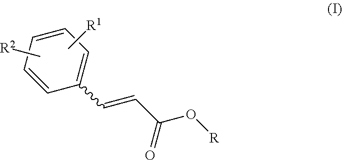
R is a C₁₋₆ alkyl group which may be substituted with 1 to 3 groups R¹, and
R¹ and R² are independently selected from the group consisting of hydrogen, halogen, C₁₋₃ alkyl and C₁₋₃ alkoxy.

33. The kit of claim 32, wherein in the cinnamic ester of formula (I) R is a C₁₋₃ alkyl group and R¹ and R² are independently selected from the group consisting of hydrogen and methyl.

34. The kit of claim 32, wherein the cinnamic ester is ethyl trans-cinnamate.

35. The kit of claim 28, wherein the clearing composition comprises from 10 to 100 vol.-% of cinnamic ester, the remainder being an optically feasible, inert organic solvent with a refractive index of about 1.5.
